(19) United States
(12) Patent Application Publication
(10) Pub. No.: US 2026/0240946 A1
(43) Pub. Date: Aug. 20, 2026

(54) Methods for Treating Premature Newborn Infants by Administration of Recombinant Human Surfactant Protein D (rhSP-D)

(71) Applicant: Airway Therapeutics, Inc., Marietta, GA (US)

(72) Inventors: Raquel Arroyo Rodriguez, Madrid (ES); Marc Salzberg, Marietta, GA (US)

(21) Appl. No.: 19/471,580

(22) PCT Filed: Nov. 1, 2024
(86) PCT No.: PCT/US2024/054089

§ 371 Date: Oct. 1, 2025

Related U.S. Application Data
(60) Provisional application No. 63/595,507, filed on Nov. 2, 2023.
(60) Provisional application No. 63/645,662, filed on May 10, 2024.

(51) Int. Cl. A61K 38/17; A61K 9/00; A61P 11/00

(52) U.S. Cl. CPC A61K 38/1732; A61K 9/0082; A61P 11/00

(57) ABSTRACT

A method for reducing the duration of mechanical ventilation in a premature infant at risk for developing bronchopulmonary dysplasia (BPD) includes administering an effective amount of recombinant human surfactant protein D (rhSP-D) to the infant. In further aspects a method for reducing the duration of days in a hospital in a premature infant at risk for developing BPD includes administering an effective amount of rhSP-D to the infant. In certain aspects a method for reducing a probability that a premature infant at risk for developing BPD will develop one or more secondary comorbidities of prematurity includes administering an effective amount of recombinant human surfactant protein D (rhSP-D) to the infant. Pharmaceutical compositions for use in the described methods and uses of a pharmaceutical composition are also described.

### FIELD OF THE DISCLOSURE

The present disclosure relates to the field of medical therapeutics, and in particular to advanced treatments aimed at improving respiratory function, reducing inflammation, clearing infection and reducing the dependency on mechanical ventilation for patients suffering from respiratory illnesses. In specific aspects, the present disclosure relates to neonatal care therapeutics, specifically to methods and compositions for reducing the duration of days in a hospital for premature infants through the administration of rhSP-D.

### BACKGROUND OF THE DISCLOSURE

Bronchopulmonary Dysplasia (BPD) is the most common complication of preterm birth and results in significant morbidity and mortality. BPD is a multifactorial, chronic pulmonary disease affecting extremely preterm infants (<29 weeks gestational age (GA)). BPD is thought to arise due to incomplete or abnormal lung development as a consequence of prematurity, injury, infection and inflammation, and it results in simplified, large alveoli leading to abnormal, inefficient gas exchange and impaired lung mechanics. Multiple pre- and postnatal risk factors influence the development of BPD, including mechanical ventilation, oxygen toxicity, intrauterine growth restriction, lung immaturity, respiratory microbial dysbiosis, vascular events such as patent ductus arteriosus (PDA), and infection. Demographic risk factors for BPD include male sex, low birth weight, impaired growth for GA and family history of asthma. BPD is associated with prolonged hospitalization in the neonatal period, growth failure, and long-term consequences such as pulmonary and neurodevelopmental impairment, lower health-related quality of life and increased healthcare costs. The median annual hospitalization cost of infants with BPD is 2.4 times higher than those without BPD in the US.

BPD remains a significant unresolved problem in preterm infants. The global incidence of BPD has been reported between 17-75% in extremely preterm infants (<29 weeks GA), with BPD incidence rates inversely proportional to GA. Advances in the clinical management of preterm babies have led to increased survival rates and a higher incidence of BPD in recent years. Current clinical management of extremely preterm neonates may involve administration of antenatal maternal steroids as a prenatal prevention strategy, post-natal surfactant therapy, post-natal steroid administration, combined surfactant and steroid therapy, caffeine therapy, vitamin A, and avoiding unnecessary mechanical ventilation. However, none of these are labelled for the treatment or prevention of BPD.

Premature infants born at a gestational age of 29 weeks or less often require extended stays in the hospital (e.g., the neonatal intensive care unit, or “NICU”) due to their underdeveloped organs and the complications associated with prematurity. These complications can include respiratory distress syndrome, bronchopulmonary dysplasia, intraventricular hemorrhage, necrotizing enterocolitis, and sepsis, among others. The prolonged stay in the NICU not only poses a risk of hospital-acquired infections and stress to the infants and their families but also significantly increases healthcare costs. Therefore, there is a need for interventions that can safely reduce the NICU stay duration by addressing the root physiological challenges faced by premature infants.

Mechanical ventilation is a primary intervention for newborns experiencing respiratory distress or failure, a condition often observed in premature births. Despite its lifesaving role, the extended use of mechanical ventilation can lead to numerous negative outcomes, such as ventilator-associated lung injury, bronchopulmonary dysplasia, chronic lung disease, pneumonia, weakened diaphragmatic function, and other increased morbidity complications. Therefore, there is a need for innovative interventions that can shorten the duration of mechanical ventilation, thereby reducing associated risks and improving outcomes for neonatal patients.

Commercial surfactant preparations are integral to lung function, in that they reduce inflammation and infection in the body while modulating the immune response to break the cycle of injury and inflammation. In so doing, commercial surfactant preparations including phospholipids such as surfactant protein B (SP-B) and surfactant protein C (SP-C) contribute to the reduction of surface tension and thereby prevent alveolar collapse, which is necessary for effective respiratory function. Conventional commercial surfactant preparations do not include surfactant protein D (SP-D). While the therapeutic potential of surfactant protein administration has been recognized in certain patient populations, methods for enhancing pulmonary health and reducing the need for prolonged mechanical support of premature infants have yet to be demonstrated.

These and other shortcomings are addressed by aspects of the present disclosure.

### SUMMARY

The present disclosure introduces a novel therapeutic method and pharmaceutical composition for the administration of rhSP-D to premature infants requiring mechanical ventilation, aimed at reducing the duration of their stay in the hospital (e.g., the NICU). Specifically, the present disclosure provides a method of reducing the duration (number of days) of mechanical ventilation required by a premature infant by administering an effective amount of rhSP-D. It further aims at reducing bronchopulmonary dysplasia (BPD). This method involves the delivery of rhSP-D to patients undergoing mechanical ventilation due to respiratory failure from any cause. The disclosure further encompasses pharmaceutical compositions comprising rhSP-D. In some aspects the rhSP-D is formulated for delivery by intratracheal, endotracheal, inhalation, or nebulization methods. The composition including rhSP-D, known as zelpultide alfa, is identified for its properties that enhance the maturation of lungs by supplementing deficient native SP-D levels in the premature lung and enhance the function of the premature lung by supplementing its deficient native SP-D levels, reduce inflammation, and improve overall health markers in premature infants, thereby reducing the duration of mechanical ventilation by facilitating earlier extubation from mechanical ventilation and facilitating earlier discharge from the hospital/NICU. In certain aspects the infant is a premature infant born at a gestational age of 29 weeks or less.

In specific aspects a method for reducing the duration of mechanical ventilation in a premature infant at risk for developing bronchopulmonary dysplasia (BPD) includes administering an effective amount of recombinant human surfactant protein D (rhSP-D) to the infant.

In further aspects a method for reducing the duration of days in a hospital in a premature infant at risk for developing BPD includes administering an effective amount of rhSP-D to the infant.

In particular aspects a method for reducing a probability that a premature infant at risk for developing BPD will develop one or more secondary comorbidities of prematurity includes administering an effective amount of recombinant human surfactant protein D (rhSP-D) to the infant.

In specific aspects the disclosure relates to the use of a pharmaceutical composition in (1) reducing the duration of mechanical ventilation in a premature infant at risk for developing bronchopulmonary dysplasia (BPD), (2) reducing the duration of days in a hospital in a premature infant at risk for developing BPD, or (3) reducing a probability that a premature infant at risk for developing BPD will develop one or more secondary comorbidities of prematurity, wherein the pharmaceutical composition comprises recombinant human surfactant protein D (rhSP-D).

### BRIEF DESCRIPTION OF THE FIGURES

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document.

FIG. 1A is a study diagram showing the study phases utilized in aspects of the disclosure. Initially, eight subjects (25-28 6/7 weeks GA) were randomized 3:1, therefore six subjects received standard of care (SOC) and up to two doses of intratracheally administered zelpultide alfa at each dose level (2 mg/kg, 4 mg/kg, or 6 mg/kg, FIG. 1A) starting with 2 mg/kg, and two subjects received SOC and up to two doses of air-sham (1 mL of air drawn into a dosing syringe for intratracheal instillation).” Doses were 24 hours apart. Twelve additional subjects (23-28 6/7 weeks GA) were randomized 3:1 to receive the highest-tolerated dose of zelpultide alfa, or air-sham, once daily for up to 7 days. Dose limiting toxicities (DLTs) were the determining factor for whether new subjects could receive a higher dose of zelpultide alfa and whether additional subjects could subsequently be enrolled in the study. Continued dosing for an individual was paused if a DLT event occurred and was referred to the DSMC.

FIG. 1B is a patient flow diagram of the 38 randomized preterm neonates included in the intent-to-treat analysis.

FIG. 2A is a graph showing time (days) on mechanical ventilation of subjects treated with zelpultide alfa versus subjects treated with air-sham at week 36 PMA (postmenstrual age). In total, 30 patients reached week 36 PMA (21 treated with zelpultide alfa, 9 treated with air-sham). There was a reduced number of days on mechanical ventilation in subjects treated with zelpultide alfa vs air-sham at week 36 PMA (17.7 days vs 25.8 days respectively).

FIG. 2B is a graph showing percentage of survivors who were receiving mechanical ventilation at each study timepoint. For zelpultide alfa, at day 1, 7, 28 and week 36 PMA there were 28, 26, 23 and 22 survivors respectively. For air-sham, there were 9 survivors at all visits. Subjects who died were excluded. A lower percentage of subjects treated with zelpultide alfa were on mechanical ventilation at week 36 PMA compared to air-sham.

FIG. 3 is Table I showing baseline demographics and characteristics for subjects included in the study.

FIG. 4 is Table II showing a summary of adverse events (AEs) and significant adverse events (SAEs). The number of patients experiencing AEs and SAEs in each treatment arm from first treatment to week 36 PMA are presented with percentages in brackets.

FIGS. 5A and 5B show a table (III.A continued onto Table III.B) of AEs reported in more than 10% of subjects in any treatment arm from first treatment to week 36 PMA. The number of patients experiencing AEs in each treatment arm are presented with percentages in brackets.

FIG. 6 is Table IV showing SAEs reported from first treatment to week 36 PMA in any treatment arm from first treatment to week 36 PMA. The number of patients experiencing SAEs in each treatment arm are presented with percentages in brackets.

FIG. 7 is Table V showing key efficacy outcomes included the incidence of BPD or death at week 36 PMA in all zelpultide alfa and air-sham treatment groups. The definition and classification of BPD was based on Jensen et al. 2019.

FIG. 8 is Table VI showing additional outcomes and comorbidities that occurred in any treatment arm from first treatment to week 36 PMA. The number of patients experiencing outcomes and comorbidities in each treatment arm are presented with percentages in brackets, except days in hospital that are presented as mean (SD). For the mean number of days in hospital, there was a recalculation for number of days in hospital excluding subjects that died. Data from the 6 subjects that died by week 36 PMA was excluded from the analysis and resulted in 87.2 days (+24.1, n=21) for the all zelpultide alfa group and 90.2 days (+21.1, n=9) for the air-sham group.

FIG. 9 is Table VII showing pharmacokinetics of zelpultide alfa (ng/mL) and air-sham showing change in SP-D blood levels from baseline at day 1 for each treatment arm.

FIGS. 10A and 10B are Tables VIII.A and VIII.B showing doses of treatment received by preterm neonates in the first phase (Table VIII.A) and second phase (Table VIII.B) of the Phase 1b trial.

FIG. 11 is Table IX showing incidence of BPD or death and days on mechanical ventilation for each treatment arm and the all zelpultide alfa group from first treatment to week 36 PMA. The number of patients experiencing BPD or death in each treatment arm are presented with percentages in brackets.

FIG. 12 is Table X showing grades of BPD or death for each treatment arm and all zelpultide alfa group from the first treatment to week 36 PMA. The number of patients with each grade of BPD or death in each treatment arm is presented with percentages in brackets.

FIG. 13 is Table XI showing mean days on mechanical ventilation for each treatment arm and the all zelpultide alfa group from first treatment to week 36 PMA. Data are shown for subjects who survived to week 36 PMA and were not lost to follow up.

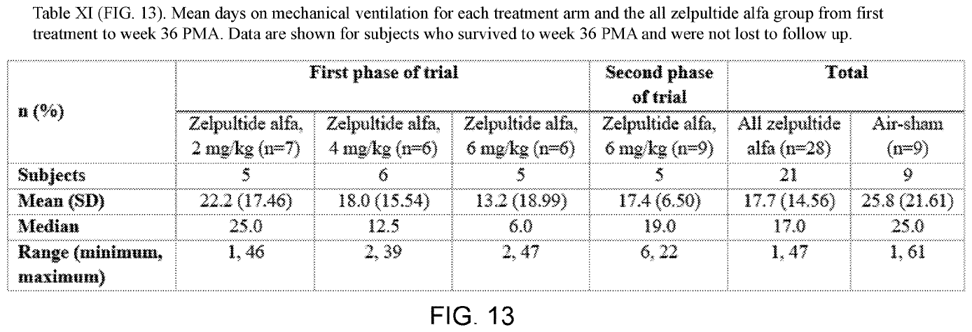

### DETAILED DESCRIPTION

Various combinations of elements of this disclosure are encompassed by this disclosure, e.g., combinations of elements from dependent claims that depend upon the same independent claim.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

All publications mentioned herein are incorporated herein by reference to disclose and describe the methods and/or materials in connection with which the publications are cited.

### Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise.

As used herein, the term “combination” is inclusive of blends, mixtures, alloys, reaction products, and the like.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the term “effective amount” refers to an amount that is sufficient to achieve the desired result. For example, an “effective amount” of a recombinant human surfactant protein D (rhSP-D) may refer to an amount that is sufficient to achieve the desired reduction in mechanical ventilation, reduction in days in the hospital, reduction in secondar comorbidities, etc. The specific level—in terms of concentration, frequency of administration, duration of administration, etc.—required as an effective amount will depend upon a variety of factors.

Bronchopulmonary dysplasia (BPD) may be assessed on an infant (and was assessed on the subjects of the study described herein) when the infant reaches 36 weeks postmenstrual age (PMA). This is the point at which the preterm infant is considered to have reached their term-equivalent age. It is calculated as the time when the gestational age (time from the first day of the last menstrual period to birth) plus the chronological age (time after birth) is 36 weeks. Criteria for assessing BPD are described in Jensen et al. (Jensen E A, Dysart K, Gantz M G, et al. The Diagnosis of Bronchopulmonary Dysplasia in Very Preterm Infants. An Evidence-based Approach. Am J Respir Crit Care Med. 2019; 200(6): 751-9) (“Jensen”), the disclosure of which is incorporated herein by this reference in its entirety. The BPD assessment for infants reaching 36 weeks PMA is to be recorded as one the following:

| BPD | |
|---|---|
| Diagnosis? | BPD Respiratory Support Diagnostic Criteria |
| No | Subject on room air (no support) at 36 weeks PMA. |
| Yes | Subject receiving respiratory support via nasal cannula |
| | (NC) at a low flow (≤2 Liters/minute, L/min), including |
| | those receiving head box oxygen, at 36 weeks PMA. |
| Yes | Subject receiving respiratory support via NC at a high |
| | flow (>2 L/min), or via nasal continuous positive |
| | airway pressure (nCPAP) and/or nasal intermittent |
| | positive pressure ventilation (NIPPV), at 36 weeks PMA. |
| Yes | Subject receiving respiratory support via invasive |
| | positive pressure ventilation (IPPV) at 36 weeks PMA. |

Jensen, et al. 2019 recommends the following BPD definition and grading categorization in which all subjects reaching the Week 36 PMA timepoint is to be recorded as one of the following:

| BPD Grading | BPD Respiratory Support Diagnostic Criteria |
|---|---|
| Not Applicable | Subject on room air (no support). |
| Grade 1 | Subject receiving respiratory support via NC ≤2 |
| | L/min, including those receiving head box oxygen. |
| Grade 2 | Subject receiving respiratory support via NC >2 |
| | L/min, or via nCPAP and/or NIPPV. |
| Grade 3 | Subject receiving respiratory support via |
| | Invasive PPV. |

Zelpultide alfa (previously AT-100) is a novel, biotechnological therapy with the active ingredient recombinant human surfactant Protein D (rhSP-D) that is in development for the prevention of BPD in preterm neonates. Current surfactant therapies for respiratory distress syndrome do not include surfactant Protein D (SP-D), which is involved in regular surfactant lipid structure and lipid recycling. SP-D helps regulate the innate immune response of the lungs by clearing infectious pathogens, limiting pulmonary inflammation and inflammatory injury. It has been hypothesized that treatment of preterm neonates with zelpultide alfa may reduce lung injury and inflammation and, therefore, will reduce time on respiratory support leading to a reduction in the incidence of BPD.

The trial described herein aimed to assess three dose levels of zelpultide alfa that maximize benefit-to-risk in extremely preterm neonates who were at high risk of developing BPD. The primary objective was to establish a safety and tolerability profile of the optimal dose of zelpultide alfa. Secondary objectives were to evaluate preliminary efficacy outcomes, including incidence of BPD or death and time on mechanical ventilation, and to evaluate the complications of prematurity. Exploratory objectives included changes in inflammatory mediators potentially associated with lung injury and BPD.

### Composition of Zelpultide Alfa

The rhSP-D used in this disclosure is synthesized through recombinant DNA technology, ensuring high purity and consistent bioactivity. The composition is formulated with a pharmaceutically acceptable buffer and other components noted below, making it suitable for administration in newborn patients. As an example, zelpultide alfa is selected as a novel recombinant version of the endogenous human protein hSP-D is synthesized and formulated for administration to premature infants. The composition includes zelpultide alfa in a pharmaceutically acceptable buffer, tailored for the sensitive physiology of premature infants. The formulation is designed for intratracheal, endotracheal, inhalation, nebulization, or potentially other administration routes suitable for neonates.

Specifically, zelpultide alfa is a recombinant version of the endogenous human protein hSP-D, a protein found to reduce inflammation and infections in the lungs while modulating the immune response to break the cycle of injury and inflammation. Zelpultide alfa is manufactured as a dry powder but is reconstituted to liquid form prior to administration, and is configured for intratracheal, endotracheal, inhalation, or nebulization administration. The liquid composition includes rhSP-D or an active fragment thereof, a buffer, a sugar, and a calcium salt. The buffer is histidine, the sugar is lactose, and the calcium salt is calcium chloride (CaCl₂)). Specifically, zelpultide alfa includes 4 mg/mL rhSP-D, 5 mM Histidine, 265 mM Lactose, and 5 mM calcium chloride. The solution has a pH of 6.0. The dosages of zelpultide alfa described herein (e.g., 2 mg/kg, 4 mg/kg and 6 mg/kg) refer to the amount of active ingredient rhSP-D administered in each dose per kilogram of the subject's birth weight.

Specific compositions including rhSP-D are described in International Publication No. WO2019191247, the disclosure of which is incorporated herein by this reference in its entirety.

### Method of Administration

The method involves administering rhSP-D to newborn patients who are on mechanical ventilation. Administration can be achieved through intratracheal, endotracheal, inhalation, or nebulization methods, with dosing regimens specifically optimized for the neonatal population based on the weight of the patient.

### Clinical Efficacy

As demonstrated in the results provided herein, clinical trials involving premature infants born at a gestational age of 29 weeks or less have demonstrated that those treated with rhSP-D, namely zelpultide alfa, exhibit a reduction in the hospital/NICU stay and mechanical ventilation duration required compared to those receiving standard care. Infants treated with zelpultide alfa demonstrated an improvement in respiratory function including a reduction of incidence and severity of BPD and in some aspects could be discharged from the hospital sooner. Further, they experienced a reduction in the incidence of complications associated with prematurity and/or mechanical ventilation, including extended steroid use, pneumonia, pulmonary hypertension, NEC, and grade 2 intraventricular hemorrhage. Overall, they had better health outcomes.

### Mechanism of Action

Zelpultide alfa enhances pulmonary health in newborns by promoting the maturation of underdeveloped lungs, promoting cellular repair mechanisms, enhancing immune function, reducing systemic inflammation, reducing infection, and improving overall lung function in premature infants. This approach addresses the primary challenges faced by premature infants, leading to improved health and quicker readiness for discharge from the hospital/NICU, and facilitates the recovery of damaged lung tissues more efficiently, leading to a faster resolution of the underlying respiratory distress or failure and, consequently, a reduced need for prolonged mechanical ventilation.

### Methods of Treating Premature Infants at Risk of Developing BPD

Aspects of the disclosure relate to methods for reducing the duration of mechanical ventilation in a premature infant at risk for developing bronchopulmonary dysplasia (BPD), including administering an effective amount of recombinant human surfactant protein D (rhSP-D) to the infant.

As demonstrated in the Examples provided herein, the median duration of mechanical ventilation for infants treated with air-sham standard of care was 25 days, and the mean duration was 25.8 days. Thus, in some aspects the reduction of duration of mechanical ventilation is at least 1 day. In further aspects the reduction of duration of mechanical ventilation is from 1 to 12 days, or at least 2 days, or at least 3 days, or at least 4 days, or at least 5 days, or at least 6 days, or at least 7 days, or at least 8 days, or up to 12 days, or up to 11 days, or up to 10 days, or up to 9 days, or up to 8 days.

In further aspects the disclosure relates to methods for reducing the duration of days in a hospital in a premature infant at risk for developing BPD, including administering an effective amount of rhSP-D to the infant.

As demonstrated in the Examples provided herein, the mean duration of days in a hospital for infants treated with air-sham standard of care was 90.2 days. Thus in particular aspects the reduction of duration days in the hospital is at least 1 day. In certain aspects the reduction of duration of days in the hospital is from 1 to 30 days, or at least 2 days, or at least 3 days, or at least 4 days, or at least 5 days, or at least 6 days, or at least 7 days, or at least 8 days, or at least 9 days, or at least 10 days, or at least 11 days, or at least 12 days, or at least 13 days, or at least 14 days, or at least 15 days, or at least 16 days, or at least 17 days, or at least 18 days, or at least 19 days, or at least 20 days, or up to 30 days, or up to 29 days, or up to 28 days, or up to 27 days, or up to 26 days, or up to 25 days, or up to 24 days, or up to 23 days, or up to 22 days, or up to 21 days, or up to 20 days.

In some aspects a method for reducing a probability that a premature infant at risk for developing BPD will develop one or more secondary comorbidities of prematurity includes administering an effective amount of recombinant human surfactant protein D (rhSP-D) to the infant.

The one or more secondary comorbidities of prematurity include, but are not limited to, steroid use, pneumonia confirmed by X-ray, retinopathy of prematurity (ROP), pulmonary hypertension, necrotizing enterocolitis (NEC), grade 2 intraventricular hemorrhage (IVH), or a combination thereof.

In further aspects the reduction in probability is at least 10%. In specific aspects the reduction in probability is at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100%. Reduction in probability may be calculated by comparing the probability that an infant receiving a standard of care treatment will develop one or more secondary comorbidities (e.g., 90%) to the probability that an infant treated with rhSP-D according to the methods described herein will develop one or more secondary comorbidities (e.g., 10%). The reduction in probability in this example would be (90−10)/90*100, or 88%.

In some aspects the rhSP-D is zelpultide alfa.

The rhSP-D may be administered intratracheally, endotracheally, by inhalation, or by nebulization.

In certain aspects the premature infant at risk for developing BPD is an infant born at a gestational age of 29 weeks or less. In further aspects the premature infant at risk for developing BPD is an infant born at a gestational age of 28 weeks or less, or 27 weeks or less, or 26 weeks or less, or 25 weeks or less, or 24 weeks or less, or 23 weeks or less, or 22 weeks or less.

In particular aspects, the premature infant at risk for developing BPD is an infant that requires respiratory support after birth. In some aspects the respiratory support is invasive respiratory support. An example of invasive respiratory support includes use of an endotracheal tube.

In further aspects the effective amount of rhSP-D includes a dosage of from 1 milligram per kilogram (mg/kg) to 10 mg/kg per day for a time period of from 1 day to 10 days. In specific aspects the dosage is 1 mg/kg per day, or 2 mg/kg per day, or 3 mg/kg per day, or 4 mg/kg per day, or 5 mg/kg per day, or 6 mg/kg per day, or 7 mg/kg per day, or 8 mg/kg per day, or 9 mg/kg per day, or 10 mg/kg per day. In particular aspects the time period is 1 day, or 2 days, or 3 days, or 4 days, or 5 days, or 6 days, or 7 days, or 8 days, or 9 days, or 10 days. As used herein a dosage of “mg/kg” means milligrams of active ingredient (e.g., rhSP-D) per kilogram of body weight of the infant at birth.

Additional aspects of the disclosure relate to a pharmaceutical composition for use in any of the methods described herein.

Further aspects of the disclosure relate to use of a pharmaceutical composition in (1) reducing the duration of mechanical ventilation in a premature infant at risk for developing bronchopulmonary dysplasia (BPD) or (2) reducing the duration of days in a hospital in a premature infant at risk for developing BPD, wherein the pharmaceutical composition includes recombinant human surfactant protein D (rhSP-D).

Yet further aspects of the disclosure relate to use of a pharmaceutical composition in (1) reducing the duration of mechanical ventilation in a premature infant at risk for developing bronchopulmonary dysplasia (BPD), (2) reducing the duration of days in a hospital in a premature infant at risk for developing BPD, or (3) reducing a probability that a premature infant at risk for developing BPD will develop one or more secondary comorbidities of prematurity, wherein the pharmaceutical composition comprises recombinant human surfactant protein D (rhSP-D).

Various combinations of elements of this disclosure are encompassed by this disclosure, e.g., combinations of elements from dependent claims that depend upon the same independent claim.

### Aspects of the Disclosure

In various aspects, the present disclosure pertains to and includes at least the following aspects.

Aspect 1. A method for reducing the duration of mechanical ventilation in a premature infant at risk for developing bronchopulmonary dysplasia (BPD), comprising administering an effective amount of recombinant human surfactant protein D (rhSP-D) to the infant.

Aspect 2. The method according to Aspect 1, wherein the reduction of duration of mechanical ventilation is at least 1 day.

Aspect 2A. The method according to Aspect 1 or 2, wherein the reduction is relative to a duration of 25 days for an infant treated with an air-sham standard of care.

Aspect 3. A method for reducing the duration of days in a hospital in a premature infant at risk for developing BPD, comprising administering an effective amount of rhSP-D to the infant.

Aspect 4. The method according to Aspect 3, wherein the reduction of days in the hospital is at least 1 day.

Aspect 4A. The method according to Aspect 3 or 4, wherein the reduction is relative to a duration of 90 days for an infant treated with an air-sham standard of care.

Aspect 5. A method for reducing a probability that a premature infant at risk for developing BPD will develop one or more secondary comorbidities of prematurity, comprising administering an effective amount of recombinant human surfactant protein D (rhSP-D) to the infant.

Aspect 6. The method of Aspect 5, wherein the one or more secondary comorbidities of prematurity comprise steroid use, pneumonia confirmed by X-ray, retinopathy of prematurity (ROP), pulmonary hypertension, necrotizing enterocolitis (NEC), grade 2 intraventricular hemorrhage (IVH), or a combination thereof.

Aspect 7. The method according to Aspect 5 or 6, wherein the reduction in probability is at least 10%.

Aspect 8. The method according to any one of Aspects 1 to 7, wherein the rhSP-D is zelpultide alfa.

Aspect 9. The method according to any one of Aspects 1 to 8, wherein the rhSP-D is administered intratracheally, endotracheally, by inhalation, or by nebulization.

Aspect 10. The method according to any one of Aspects 1 to 9, wherein the premature infant at risk for developing BPD is an infant born at a gestational age of 29 weeks or less.

Aspect 11. The method according to any one of Aspects 1 to 10, wherein the premature infant at risk for developing BPD is an infant that requires respiratory support after birth.

Aspect 12. The method according to Aspect 11, wherein the respiratory support is invasive respiratory support.

Aspect 13. The method according to Aspect 12, wherein the invasive respiratory support comprises use of an endotracheal tube.

Aspect 14. The method according to any one of Aspects 1 to 13, wherein the effective amount of rhSP-D comprises a dosage of from 1 milligram per kilogram (mg/kg) to 10 mg/kg per day for a time period of from 1 day to 10 days.

Aspect 15. A pharmaceutical composition for use in the method of any one of Aspects 1 to 14.

Aspect 16. Use of a pharmaceutical composition in (1) reducing the duration of mechanical ventilation in a premature infant at risk for developing bronchopulmonary dysplasia (BPD), (2) reducing the duration of days in a hospital in a premature infant at risk for developing BPD, or (3) reducing a probability that a premature infant at risk for developing BPD will develop one or more secondary comorbidities of prematurity,
wherein the pharmaceutical composition comprises recombinant human surfactant protein D (rhSP-D).

Aspect 17. The use according to Aspect 16, wherein the reduction of duration of mechanical ventilation is at least 1 day.

Aspect 18. The use according to Aspect 16 or 17, wherein the reduction of duration of days in the hospital is at least 1 day.

Aspect 19. The use according to any one of Aspects 16 to 18, wherein the one or more secondary comorbidities of prematurity comprise steroid use, pneumonia confirmed by X-ray, retinopathy of prematurity (ROP), pulmonary hypertension, necrotizing enterocolitis (NEC), grade 2 intraventricular hemorrhage (IVH), or a combination thereof.

Aspect 20. The use according to any one of Aspects 16 to 19, wherein the reduction in probability is at least 10%.

Aspect 21. The use according to any one of Aspects 16 to 20, wherein the rhSP-D is zelpultide alfa.

Aspect 22. The use according to any one of Aspects 16 to 21, wherein the rhSP-D is administered intratracheally, endotracheally, by inhalation, or by nebulization.

Aspect 23. The use according to any one of Aspects 16 to 22, wherein the premature infant at risk for developing BPD is an infant born at a gestational age of 29 weeks or less.

Aspect 24. The use according to any one of Aspects 16 to 23, wherein the premature infant at risk for developing BPD is an infant that requires respiratory support after birth.

Aspect 25. The use according to Aspect 24, wherein the respiratory support is invasive respiratory support.

Aspect 26. The use according to Aspect 25, wherein the invasive respiratory support comprises use of an endotracheal tube.

Aspect 27. The use according to any one of Aspects 16 to 26, wherein rhSP-D is administered at a dosage of from 1 milligram per kilogram (mg/kg) to 10 mg/kg per day for a time period of from 1 day to 10 days.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. Unless indicated otherwise, percentages referring to a composition are in terms of wt %.

There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

The present examples relate to a study entitled “A Phase 1b Randomized, Multicenter, Dose-Determination Trial Of Zelpultide Alfa (rhSP-D) In Preterm Neonates At High Risk Of Developing Bronchopulmonary Dysplasia.”

The objective of the Phase 1b Study (clinical safety study) was to assess the safety and tolerability of different doses of zelpultide alfa versus air-sham added to standard of care in preterm neonates at risk of bronchopulmonary dysplasia (BPD). Efficacy was a secondary outcome.

The study was designed as a randomized, double-blind, dose-determination study that enrolled intubated, mechanically-ventilated preterm neonates who required ≥1 surfactant treatment within 96 hours of birth. Initially, eight subjects (25-28 6/7 weeks gestational age, GA) were randomized 3:1 to receive up to two doses of intratracheal zelpultide alfa at each dosing level (2, 4 or 6 mg/kg) or air-sham, 24 hours apart. Twelve additional subjects (23-28 6/7 weeks GA) were randomized 3:1 to receive the highest-tolerated dose of zelpultide alfa, or air-sham, once daily for up to 7 days.

Thirty-seven subjects were randomized and treated. Zelpultide alfa 6 mg/kg was safe and well tolerated. 92.9% of zelpultide alfa subjects versus 100.0% air-sham experienced ≥1 adverse event. Mortality was 21%, with all cases in the zelpultide alfa group, although no deaths were related to study drug. Zelpultide alfa subjects had better outcomes versus air-sham with lower incidence of BPD (32.1% vs 66.7%), grade 2 or 3 BPD or death (39% vs 56%) and time on mechanical ventilation (17.7 vs 25.8 days).

As explained in further detail, the study supports the safety and tolerability of zelpultide alfa, confirming 6 mg/kg (≤7 days) as safe and well tolerated. Efficacy outcomes improved with zelpultide alfa, indicating this novel drug is a promising therapy for the prevention of BPD.

Registry numbers for the clinical trial are NCT04662151 and EudraCT 2021-005752-10.

The following abbreviations are used in reporting the results:

List of abbreviations

| | |
|---|---|
| AE | Adverse event |
| BPD | Bronchopulmonary dysplasia |
| DLT | Dose limiting toxicity |
| DSMC | Data Safety Monitoring Committee |
| ETT | Endotracheal tube |
| GA | Gestational age |
| IVH | Intraventricular hemorrhage |
| NEC | Necrotizing enterocolitis |
| NICU | Neonatal intensive care unit |
| PDA | Patent ductus arteriosus |
| PMA | Postmenstrual age |
| rhSP-D | Recombinant human Surfactant Protein D |
| ROP | Retinopathy of prematurity |
| SAE | Serious adverse event |
| SD | Standard deviation |
| SP-D | Surfactant Protein D |
| SOC | Standard of care |
| US | United States |

### Trial Design

The study was conducted in 12 sites in the United States and 10 in Spain. The trial was designed, conducted, recorded, and reported in compliance with the principles of Good Clinical Practice guidelines, and monitored by an appropriate Data Safety Monitoring Committee (DSMC). The study evaluated the safety and tolerability of zelpultide alfa in preterm neonates at high risk of developing BPD, compared to air-sham. The main purpose of this trial was to establish the safety profile of the optimal dose of zelpultide alfa.

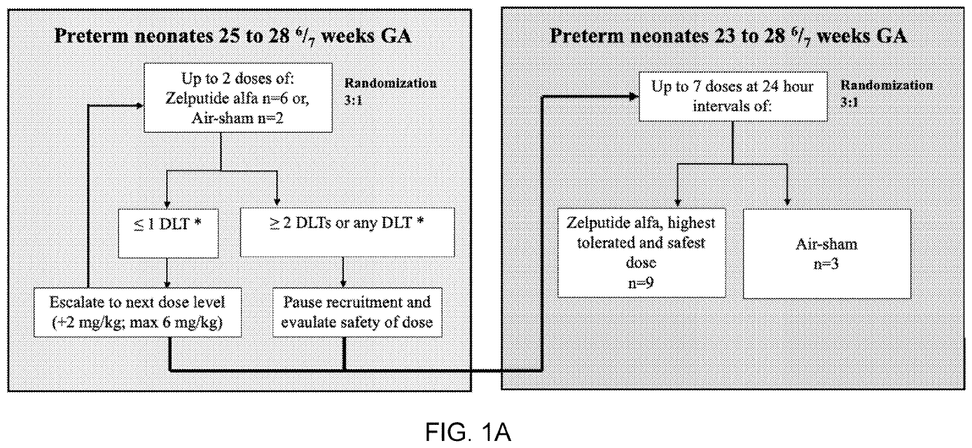

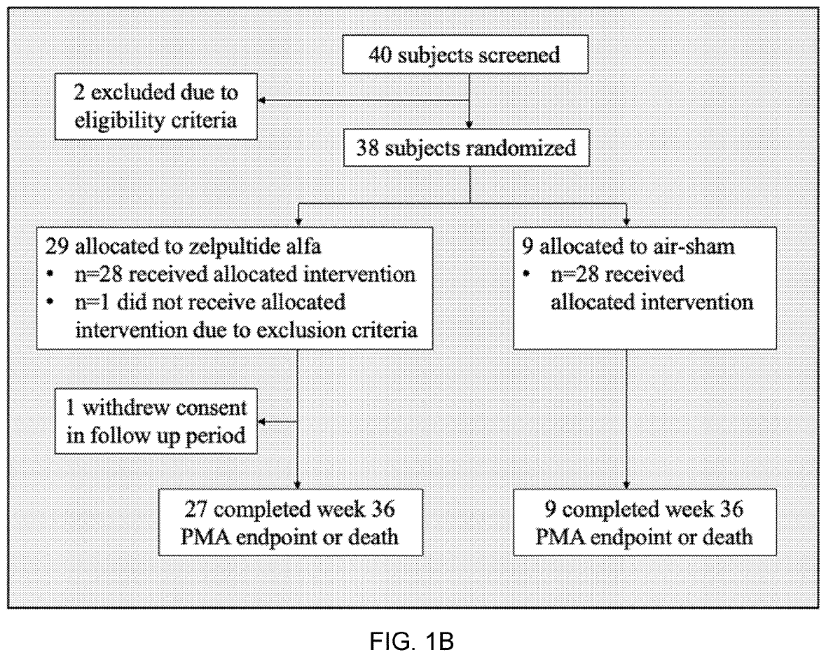

This study had two phases. In the first phase, extremely preterm neonates (born between 25 and 28 6/7 weeks GA) were randomized 3:1 in a sequential collective cohort approach following classical dose-determining study design, with six who received standard of care (SOC) and intratracheally administered zelpultide alfa at each dose level (2 mg/kg, 4 mg/kg, or 6 mg/kg, see FIG. 1A) starting with 2 mg/kg, and two who received SOC and air-sham (1 mL of air drawn into a dosing syringe for intratracheal instillation). Randomized subjects received up to two doses of study treatment, 24 hours (+1 hour) apart, if still intubated at the time of the second dose. The first dose of zelpultide alfa or air-sham was administered through the endotracheal tube (ETT) at least 15 minutes after Curosurf® surfactant treatment and within 96 hours of birth. In the second phase, once the highest-tolerated dose was confirmed, extremely preterm neonates (born between 23 weeks and 28 6/7 weeks GA) were enrolled concurrently and randomized 3:1 with nine who received zelpultide alfa and three who received air-sham. Subjects received up to seven doses of zelpultide alfa or air-sham at approximately 24-hour (±1 hour) intervals if still intubated per standard of care.

Dose Limiting Toxicities (DLTs) were the determining factor for whether new subjects could receive a higher dose of zelpultide alfa and whether additional subjects could subsequently be enrolled in the study. Continued dosing for a subject was paused if a DLT event occurred and was referred to the DSMC. All subjects received SOC, including but not limited to, pulmonary surfactant, respiratory support and nutrition, as per local hospital clinical guidelines. Study participation did not influence SOC or change the clinical management of subjects enrolled. Follow-up occurred at day 28, week 36 PMA, hospital discharge, and months 6 and 12 of life for all subjects (6- and 12-month data are not reported in this publication). The healthcare team, investigators, subjects and their parents were blinded to randomization. However, due to the nature of intratracheal treatment in the NICU, staff responsible for dose preparation and administration of zelpultide alfa and air-sham were aware of randomization assignment. These individuals conducted the dosing process out of sight and were requested not to disclose each subject's randomization to blinded staff.

### Study Subjects

In the first phase of the trial (which included dose escalation and up to 2 days of treatment), 24 subjects born between 25 and 28 6/7 weeks GA were planned to be enrolled. In the second phase of the trial (which tested up to 7 days of treatment), 12 subjects born between 23 and 28 6/7 weeks GA were planned to be enrolled. Due to an error of the randomization system, one additional subject was randomized to the first phase 2 mg/kg zelpultide alfa treatment group. After randomization but prior to treatment administration, one subject no longer met the eligibility criteria so was withdrawn from the study and replaced, thus, a total of 38 subjects were randomized and 37 received treatment (FIG. 1B). Eligibility criteria included endotracheal intubation, mechanical ventilation, and receipt at least one dose of surfactant treatment (Curosurf®) as part of SOC after birth; the study population was limited to subjects who received Curosurf® to reduce variability related to surfactant choice. Subjects were excluded if birth weight was <400 g or >1,800 g, and they had any major congenital abnormalities impacting cardiovascular and pulmonary function. Subjects were also excluded if their birth mother had Hepatitis B, C, or E, HIV, was receiving chemotherapy, had known active cytomegalovirus, COVID-19 or sexually transmitted infection, or a history of alcohol or drug abuse. The thirty-seven subjects who were randomized and treated were included in the intention-to-treat population.

### Outcomes

The primary outcome was safety and tolerability of zelpultide alfa. Safety outcomes included incidence of adverse events (AEs), serious adverse events (SAEs), hematology, and serum chemistry lab abnormalities. The DSMC evaluated AEs and DLTs for any zelpultide alfa-related safety implications and made recommendations regarding escalation of dose levels and subsequent enrolment, including termination of enrolment if appropriate. DLTs were distinguished from AEs if all of the following criteria were met: if the AE was possibly, probably, or directly related to zelpultide alfa administration; if the AE occurred at any time between the initial and last dose of zelpultide alfa +72 hours; the AE was not directly related to surfactant administration or ETT placement error; the AE caused any of the following that were unstable after medical intervention or resulted in death: hemodynamic instability, shock or severe hypertension, partial pressure of carbon dioxide <25 or >65 mmHg, apnea, ETT/airway blockage that required immediate removal of ETT, hypoxia, extubation/reintubation resulting in death, or anaphylaxis.

Secondary outcomes evaluated preliminary efficacy outcomes and complications of prematurity. Key efficacy outcomes included the incidence of BPD or death at week 36 PMA and time (days) on mechanical ventilation from birth to 36 weeks PMA. The definition and classification of BPD was based on Jensen et al. 2019 (Jensen E A, Dysart K, Gantz M G, et al. The Diagnosis of Bronchopulmonary Dysplasia in Very Preterm Infants. An Evidence-based Approach. Am J Respir Crit Care Med. 2019; 200(6):751-9). Days on mechanical ventilation were defined as ≥12 hours of mechanical ventilation within a calendar day. Other secondary outcomes included steroid use, number of days in hospital, immunogenicity, pharmacokinetic analysis, pneumonia, blood infection, incidence retinopathy of prematurity (ROP), pulmonary hypertension, PDA, necrotizing enterocolitis (NEC), or grade 2 intraventricular hemorrhage (IVH). Exploratory endpoints included changes in inflammatory mediators potentially associated with lung injury and BPD through 36 weeks.

Given this was a phase 1b study, the sample size was not statistically powered for safety and efficacy-related outcomes, thus, no inferential statistics were planned, and analyses of safety and efficacy endpoints were descriptive only. Continuous variables were summarized by mean, standard deviation (SD), median, minimum, and maximum. Categorical variables were reported by the number and percentage of subjects. Baseline results were defined as the closest measurements taken prior to the first dose of study medication.

### Results

The study was performed in two parts: the initial study examined the primary endpoint of safety and tolerability of zelpultide alfa; study completion including the long-term follow-up endpoints (up to 12 months of life). Of the extremely preterm neonates who received treatment (in the first phase, zelpultide alfa 2 mg/kg n=7, zelpultide alfa 4 mg/kg n=6, zelpultide alfa 6 mg/kg n=6, and air-sham n=6; in the second phase zelpultide alfa 6 mg/kg n=9, and air-sham n=3), 36 completed the week 36 PMA or death endpoint (FIG. 1B). One subject discontinued the study before week 36 PMA due to withdrawal of consent (in the first phase, zelpultide alfa 2 mg/kg n=1).

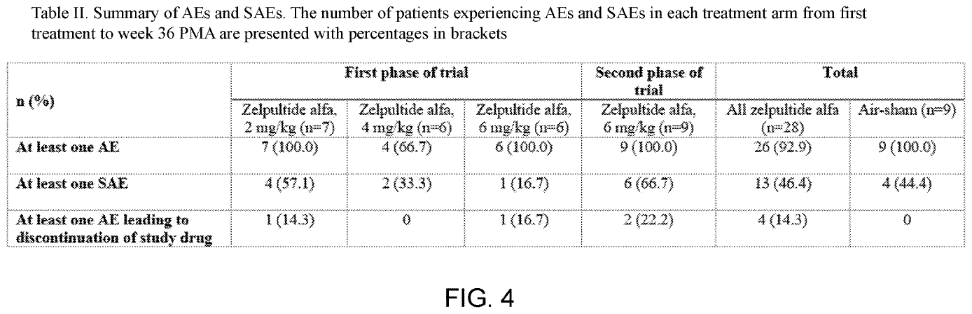

Most subjects were male, non-Hispanic/Latino and Caucasian. Mean baseline length/height, weight, and head circumference were generally comparable across groups (see Table I, FIG. 3). The mean GA at baseline in the air-sham treatment arm was slightly younger compared to zelpultide alfa; however, the median age was numerically comparable between the two groups (air-sham: 25.6 [min, max, 24, 28]; zelpultide alfa: 26.1 [min, max, 23, 29]). The zelpultide alfa treatment arm also had a higher incidence of Black/African American babies (17.9%) and congenital abnormalities (32.1%) compared with the air-sham group (11.1% for both).

### Safety Outcomes

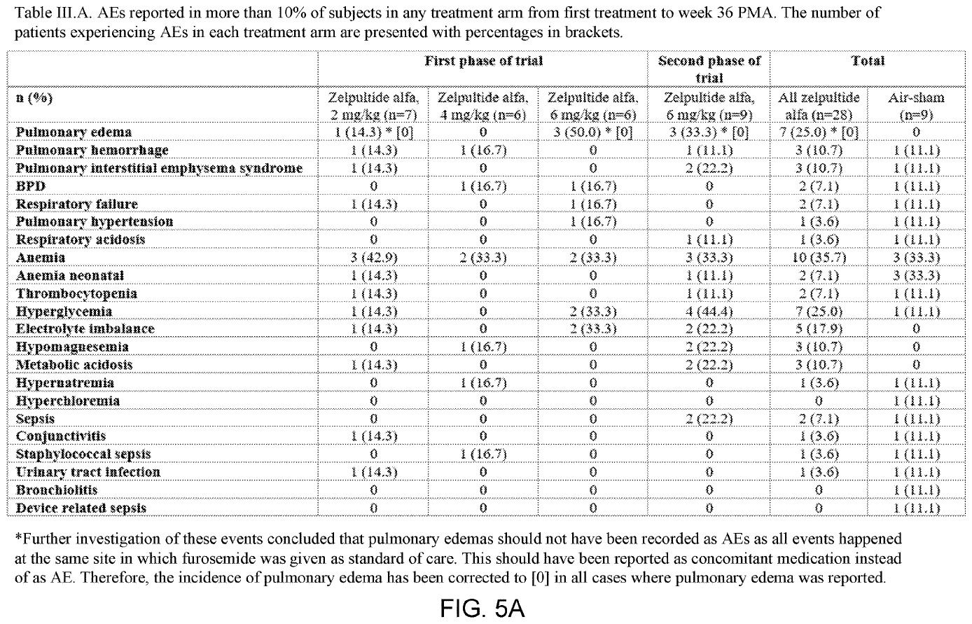

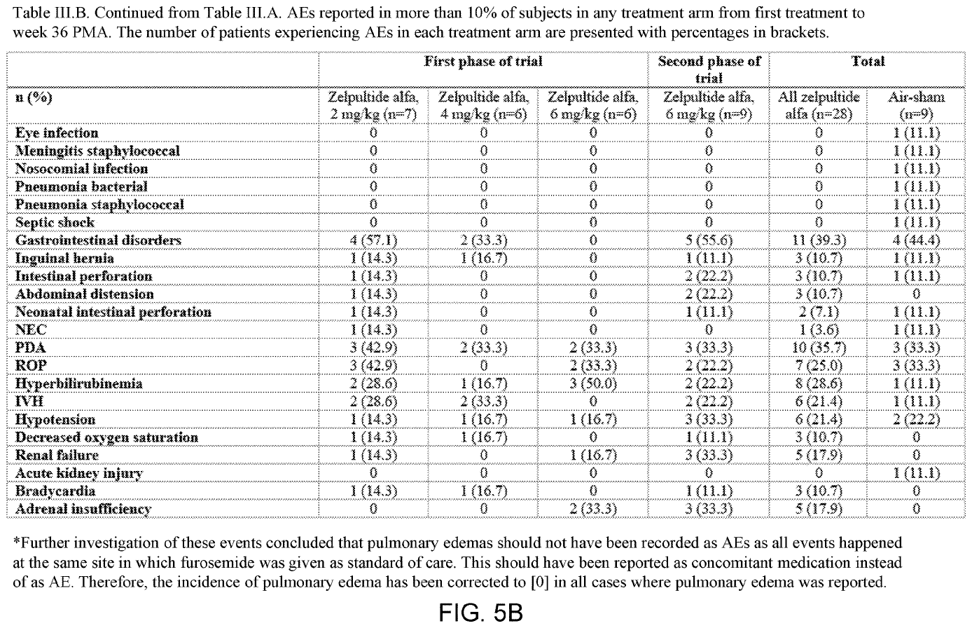

Treatment with zelpultide alfa was safe and well tolerated. Dose escalation proceeded to the highest dose, with no DLTs reported. Thus, 6 mg/kg was chosen as the highest-tolerated, safest dose for the second phase of the study. In total, 92.9% of all subjects treated with zelpultide alfa experienced at least one AE compared with 100% of those treated with air-sham (Table II, FIG. 4). Across all treatment groups, the most frequently reported AEs were anemia, PDA, ROP, hyperbilirubinemia, hyperglycemia, and hypotension (Tables III.A and III.B, FIGS. 5A and 5B).

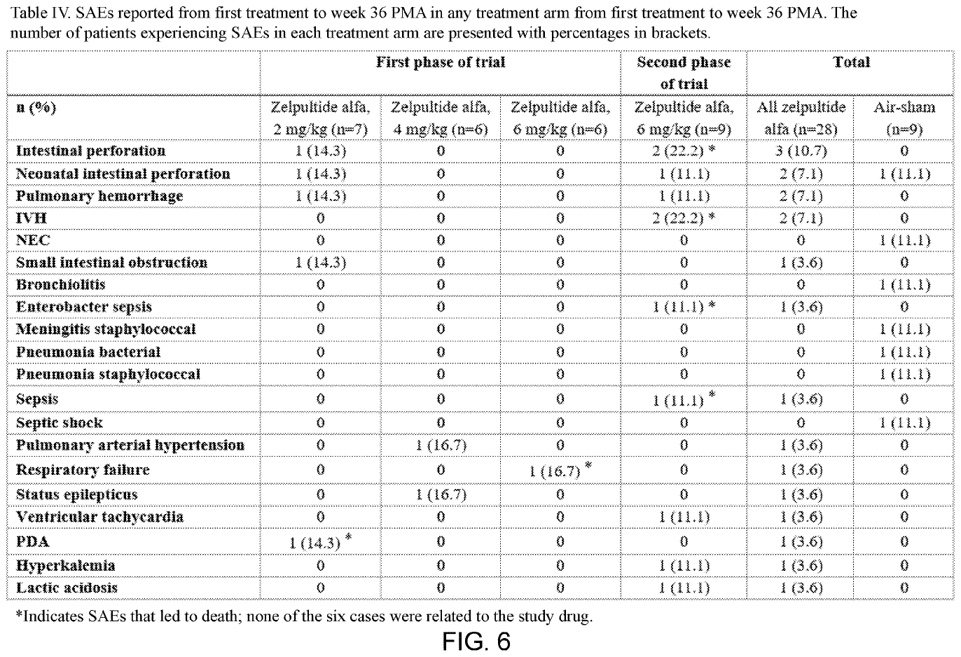

Four subjects in the zelpultide alfa arms of the first phase (2 mg/kg, n=2; 4 mg/kg, n=2) and three subjects in the second phase of the trial (6 mg/kg up to seven doses, n=3) experienced a total of nine AEs that were considered related to zelpultide alfa. Eight of these AEs were considered possibly related to treatment and one was considered probably related to treatment (hypoxia). No AEs were reported that were related to treatment in the zelpultide alfa 6 mg/kg in the first phase of the trial or air-sham treatment groups. Seventeen subjects experienced 26 SAEs. Only two SAEs were considered possibly related to treatment by the investigator (both were pulmonary hemorrhage cases); however, these were not considered related by the study and Sponsor medical monitor due to the nature of preterm birth and associated complications. The other 24 SAEs were not related to the study drug. Six AEs led to death in this study; none were considered related to treatment by the investigators, study medical monitor or Sponsor medical monitor. Five were due to non-respiratory conditions including Enterobacter sepsis, sepsis, PDA, intestinal perforation, and IVH (Table IV, FIG. 6). One subject died due to respiratory failure. Four subjects treated with zelpultide alfa experienced SAEs that led to withdrawal of the study drug (pulmonary hemorrhage, respiratory failure, Enterobacter sepsis, and IVH); the majority of these (n=3) were considered unrelated to treatment.

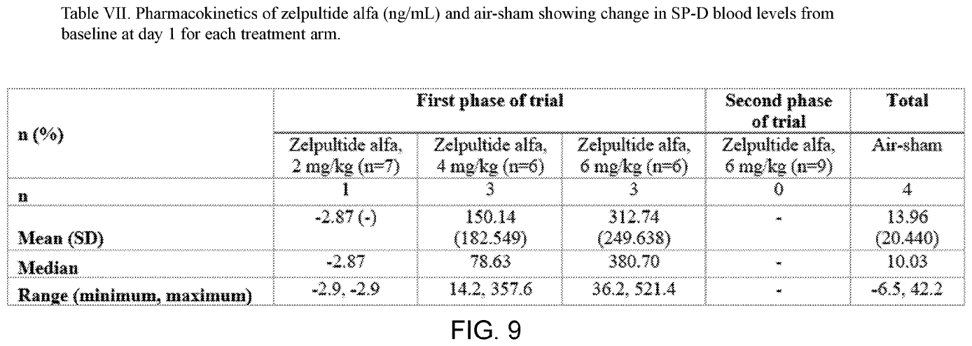

In terms of immunogenicity, as determined by the presence of anti-SP-D antibodies, no anti-zelpultide alfa antibodies developed in any subjects treated with zelpultide alfa through week 36 PMA. In the pharmacokinetic analysis, dose-dependent increases in blood SP-D concentrations were observed in subjects treated with zelpultide alfa in the 24-hours after the first treatment dose compared to baseline, and these were highest with the administration of the 6 mg/kg dose (Table VII, FIG. 9).

Given the relatively low incidence of AEs, SAEs, no zelpultide alfa-related AEs that led to death, and no reported DLTs, treatment with zelpultide alfa was considered to be safe and well tolerated.

### Efficacy Outcomes

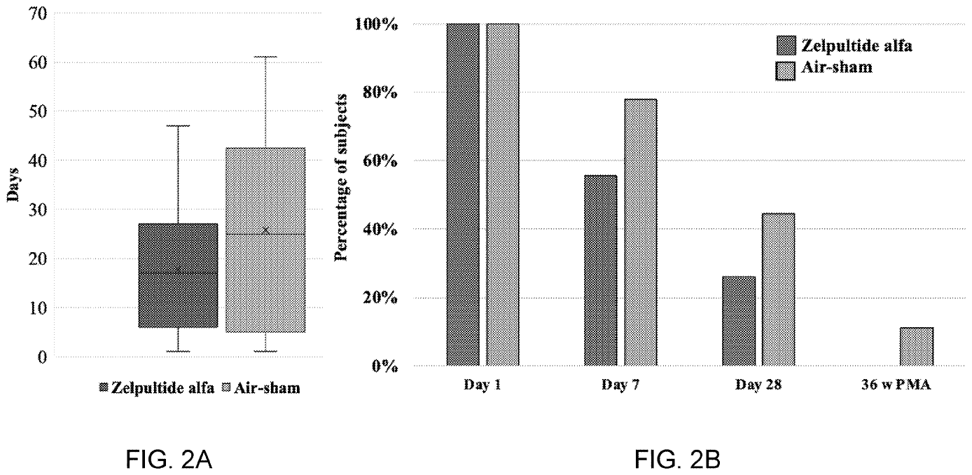

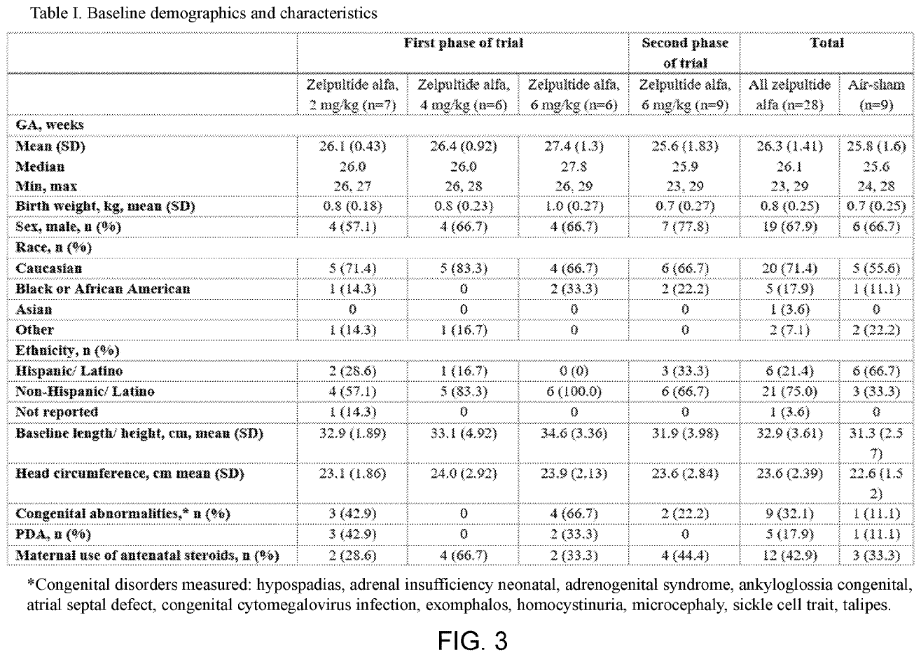

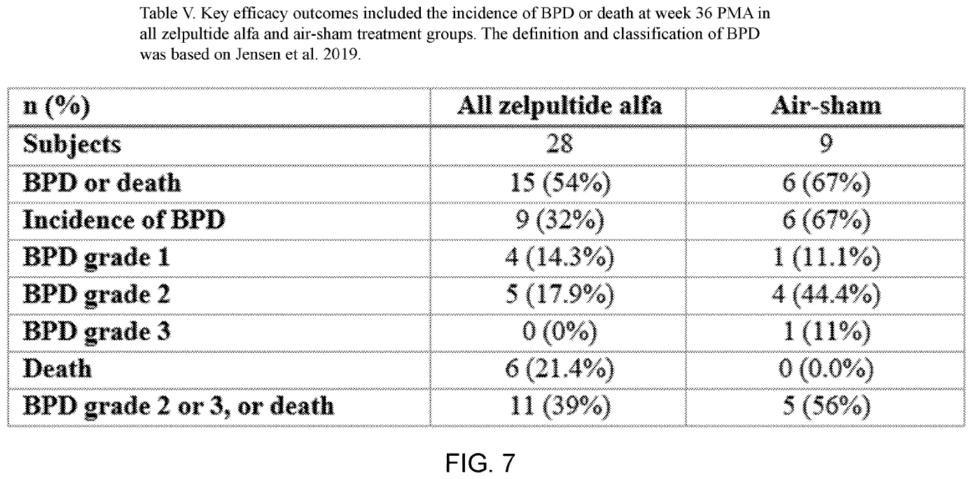

Zelpultide alfa improved the overall outcome of extremely preterm neonates in multiple clinically meaningful respiratory outcomes, including the reduction of BPD and time on mechanical ventilation. Subjects treated with zelpultide alfa demonstrated a lower incidence of BPD (32.1% vs 66.7%), lower incidence of BPD or death (53.6% vs 66.7%), lower incidence of grade 2 or 3 BPD or death (39% vs 56%), no cases of grade 3 BPD (0% vs 11.1%) (Table V, FIG. 7), reduced time on mechanical ventilation (mean: 17.7 days vs 25.8 days, FIG. 2A) and lower percentage of subjects on mechanical ventilation at week 36 PMA compared to air-sham (FIG. 2B).

### Additional Outcomes

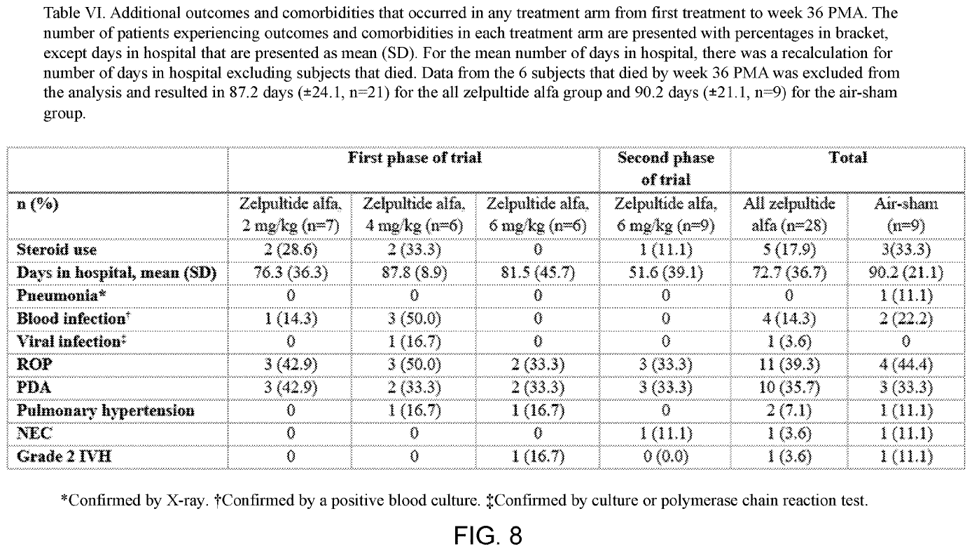

Several notable findings from additional analyses of secondary and exploratory outcomes were observed in this phase 1b study. The use of steroids, defined as any steroid concomitant medication used to treat postnatal lung disease, was numerically lower in subjects treated with zelpultide alfa compared to air-sham (17.9% vs 33.3%, Table VI, FIG. 8). The mean number of days in hospital between baseline and discharge, excluding the data from the six subjects that died by week 36 PMA resulted in 87.2 days (±24.1, n=21) for the all zelpultide alfa group and 90.2 days (±21.1, n=9) for the air-sham group. The incidence of ROP, pulmonary hypertension and NEC were also numerically lower in subjects treated with zelpultide alfa compared with air-sham (39.3% vs 44.4%, 7.1% vs 11.1%, and 3.6% vs 11.1%, respectively, Table VI).

### Discussion

In this phase 1b, randomized clinical trial, the highest tested dose of intratracheal zelpultide alfa (6 mg/kg administered for up to 7 days) was shown to be safe and well tolerated when added to SOC for the treatment of extremely preterm neonates at high risk of developing BPD. There were no DLTs reported in subjects who received zelpultide alfa. The DSMC assessed the 6 mg/kg dose as safe and recommended it for progression into future studies. Multiple clinically meaningful respiratory outcomes also improved in subjects treated with zelpultide alfa compared to air-sham. The incidence of BPD, BPD or death, grade 2 or 3 BPD or death, and time on mechanical ventilation at week 36 PMA were numerically lower in subjects treated with zelpultide alfa compared to air-sham. These improved outcomes were accompanied by a lower incidence of steroid use, reduced ROP, pulmonary hypertension and NEC at week 36 PMA. The zelpultide alfa treatment arm included a higher percentage of Black/African American babies. Racial and ethnic disparities in neonatal comorbidities and mortality have been reported in the literature, including higher infant mortality associated with Black/African American race. However, findings from different studies are inconclusive and the contributing factors for these racial and ethnic disparities have not been fully identified. Nevertheless, considering this as a potential factor for poor prognosis, the positive outcomes associated with zelpultide alfa could be considered more notable.

Since there are no gold standard outcome measurements for improved pulmonary function in extremely preterm neonates, the secondary efficacy outcomes were designed to determine if zelpultide alfa therapy leads to a change in the combined outcome of BPD and mortality, and if zelpultide alfa therapy affects time on respiratory support through 36 weeks PMA. Grade 2 and 3 BPD (moderate to severe) was chosen as an outcome as babies in these categories have the highest risk of extensive morbidities, worst long-term outcomes and significant burden on intensive care services. Importantly, there was no incidence of grade 3 BPD in the twenty-eight subjects who were treated with zelpultide alfa. The data also showed a relative risk reduction of grade 2 or 3 BPD or death of 30% for subjects treated with zelpultide alfa. An additional analysis was performed excluding deaths before 14 days of life, which is a meaningful analysis due to the high rate of mortality within this period as a consequence of the comorbidities associated with prematurity. Zelpultide alfa showed a relative risk reduction of grades 2 or 3 BPD or death of 55% in babies surviving >14 days, suggesting more notable improvement in the zelpultide alfa-treated arm compared to air-sham.

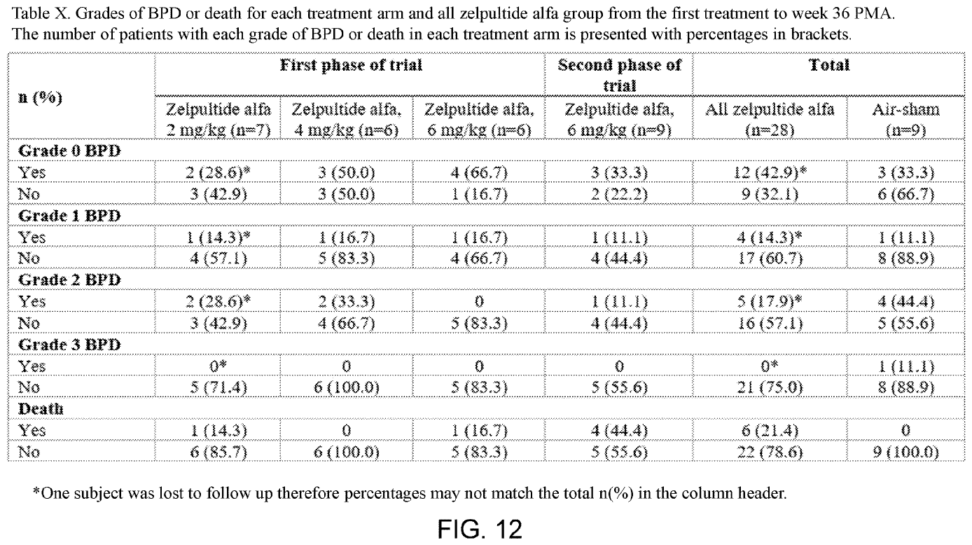

In recent years, there has been a focus on avoiding unnecessary invasive ventilation in preterm neonates to prevent long-term lung injury. Although ventilation strategies have improved, mechanically ventilated lungs are still exposed to artificial overextension driven by the pressure and tidal volumes set on ventilators. This increases lung tissue injury, blunts secondary alveolar septation, increases the risk of ventilatory associated pneumonia, and activates pro-inflammatory cascades, increasing the risk of BPD; thus it is desirable to achieve any reduction in the time spent on mechanical ventilation. Therefore, time on mechanical ventilation was included as a secondary endpoint to determine if zelpultide alfa leads to an improvement in respiratory function that reduces the need for invasive mechanical ventilation. Zelpultide alfa reduced the time on mechanical ventilation by approximately 8 days compared to air-sham (FIGS. 2A and 2B and Table XI (FIG. 12)).

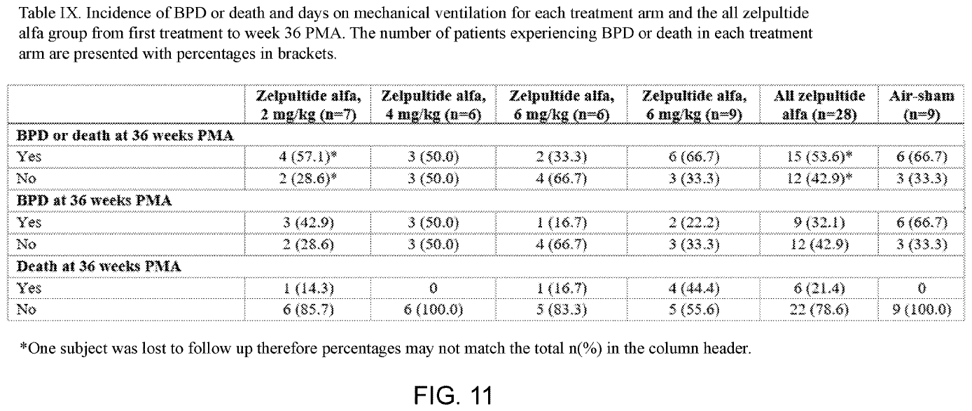

As shown in Table IX (FIG. 11), more deaths were observed in the zelpultide alfa 6 mg/kg group in the second phase of the trial, potentially because the second phase allowed enrollment of the most premature babies from 23 weeks GA. The incidence of death in the zelpultide alfa group was 21.4%, consistent with recently published mortality rates of between 15-50% in preterm neonates. Of note, most deaths occurred in younger babies (≤25 weeks GA), which is consistent with conventional knowledge that subjects in this age group have lower survival rates, and 5 of 6 cases were not related to respiratory events (only one death was due to respiratory failure and this subject also had an underlying PDA). Conversely, there were no deaths in the air-sham group during the observation period. This may be considered a statistical anomaly, likely due to the small sample size in the air-sham group (n=9) compared to 28 subjects in the zelpultide alfa group and the 3:1 randomization scheme which increases the chance of events occurring in the larger zelpultide alfa group. A small sample size was appropriate for this phase 1b study given the primary outcome was safety, although this limited the assessment of statistical significance for efficacy outcomes.

In terms of exploratory endpoints, levels of pro-inflammatory and anti-inflammatory cytokines were measured as inflammation plays a key role in the development and progression of lung injury and BPD and zelpultide alfa could have a role in modulating these inflammatory pathways. Unfortunately, inflammatory biomarker data were limited due to ethics guideline-based restrictions on the volume of blood that can be withdrawn from extremely preterm neonates and a comparison with air-sham was not possible due to non-uniform values at baseline. The limited data and baseline variability precluded the ability to reach clinically relevant conclusions for this outcome.

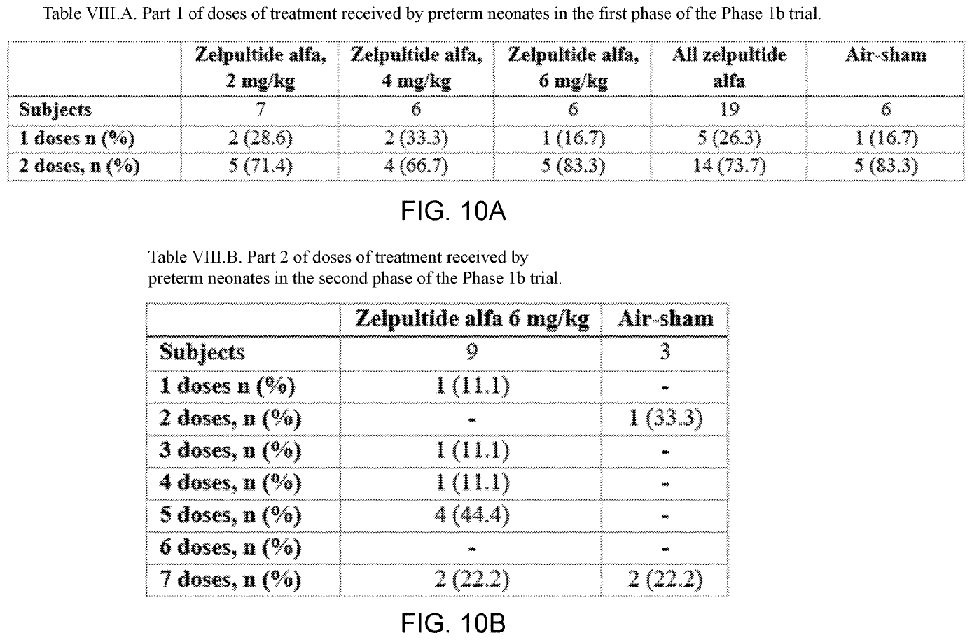

There are several limitations of this trial relating to its design. A blind clinical trial is the gold standard for limiting bias in study design. However, blinding treatment in a NICU is challenging when using an intratracheally administered product, such as zelpultide alfa, within a limited time period. The use of a placebo also limits bias in any study design. However, placebo administered intratracheally in the NICU setting is not ethical or practical, therefore, SOC with air-sham was chosen as the comparator in this trial. Although air-sham as a comparator is not completely without risks associated with intratracheal use, the risks to subjects were considered minimal. Thus, individuals who prepared and administered the dose were aware of treatment; however, all other NICU healthcare staff, Investigators, subjects and their parents intended to remain blinded to treatment. In the last decade, there has been a tendency towards early extubation and earlier transition to non-invasive ventilation. The number of administrations of treatment that a subject could receive was determined by the study protocol and it was dependent on the presence of an ETT for mechanical ventilation as per SOC. Thus, there was variability in the number of treatment administrations received by each subject, depending on when subjects were extubated, as per SOC. In the second phase, where treatment could be administered for up to 7 days, over 66% of subjects received ≥5 administrations (Tables VIII.A and VIII.B, FIGS. 10A and 10B). This limitation was difficult to overcome because the study aimed not to interfere with SOC in the respiratory management of the subjects.

### CONCLUSION

Intratracheally administered zelpultide alfa for extremely preterm neonates at a dose of 6 mg/kg for up to 7 days was established as a safe and tolerated dose, and is the dose recommended by the DSMC for future studies based on the observed safety profile. Although the study was not powered for efficacy, the endpoints that assessed efficacy improved following zelpultide alfa treatment, showing a reduction in BPD or death incidence and days on mechanical ventilation. This confirms the promising clinical profile of zelpultide alfa for the prevention of BPD, especially given the challenges and co-morbidities of extremely preterm neonates at study entry.

The above description is intended to be illustrative, and not restrictive. For example, the above-described examples (or one or more aspects thereof) may be used in combination with each other. Other aspects can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed aspect. Thus, the following claims are hereby incorporated into the Detailed Description as examples or aspects, with each claim standing on its own as a separate aspect, and it is contemplated that such aspects can be combined with each other in various combinations or permutations. The scope of the disclosure should be determined with reference to the appended claims, along with the full scope of equivalents to which such claims are entitled.

1. A method for reducing the duration of mechanical ventilation in a premature infant at risk for developing bronchopulmonary dysplasia (BPD), comprising administering an effective amount of recombinant human surfactant protein D (rhSP-D) to the infant.

2. The method according to claim 1, wherein the reduction of duration of mechanical ventilation is at least 1 day.

3. A method for reducing the duration of days in a hospital in a premature infant at risk for developing BPD, comprising administering an effective amount of rhSP-D to the infant.

4. The method according to claim 3, wherein the reduction of duration of days in the hospital is at least 1 day.

5. A method for reducing a probability that a premature infant at risk for developing BPD will develop one or more secondary comorbidities of prematurity, comprising administering an effective amount of recombinant human surfactant protein D (rhSP-D) to the infant.

6. The method of claim 5, wherein the one or more secondary comorbidities of prematurity comprise steroid use, pneumonia confirmed by X-ray, retinopathy of prematurity (ROP), pulmonary hypertension, necrotizing enterocolitis (NEC), grade 2 intraventricular hemorrhage (IVH), or a combination thereof.

7. The method according to claim 5, wherein the reduction in probability is at least 10%.

8. The method according to claim 1, wherein the rhSP-D is zelpultide alfa.

9. The method according to claim 1, wherein the rhSP-D is administered intratracheally, endotracheally, by inhalation, or by nebulization.

10. The method according to claim 1, wherein the premature infant at risk for developing BPD is an infant born at a gestational age of 29 weeks or less.

11. The method according to claim 1, wherein the premature infant at risk for developing BPD is an infant that requires respiratory support after birth.

12. The method according to claim 11, wherein the respiratory support is invasive respiratory support.

13. The method according to claim 12, wherein the invasive respiratory support comprises use of an endotracheal tube.

14. The method according to claim 1, wherein the effective amount of rhSP-D comprises a dosage of from 1 milligram per kilogram (mg/kg) to 10 mg/kg per day for a time period of from 1 day to 10 days.

15. A pharmaceutical composition for use in the method of claim 1.

16. Use of a pharmaceutical composition in (1) reducing the duration of mechanical ventilation in a premature infant at risk for developing bronchopulmonary dysplasia (BPD), (2) reducing the duration of days in a hospital in a premature infant at risk for developing BPD, or (3) reducing a probability that a premature infant at risk for developing BPD will develop one or more secondary comorbidities of prematurity,
wherein the pharmaceutical composition comprises recombinant human surfactant protein D (rhSP-D).

17. The use according to claim 16, wherein the reduction of duration of mechanical ventilation is at least 1 day.

18. The use according to claim 16, wherein the reduction of duration of days in the hospital is at least 1 day.

19. The use according to claim 16, wherein the one or more secondary comorbidities of prematurity comprise steroid use, pneumonia confirmed by X-ray, retinopathy of prematurity (ROP), pulmonary hypertension, necrotizing enterocolitis (NEC), grade 2 intraventricular hemorrhage (IVH), or a combination thereof.

20. The use according to claim 16, wherein the reduction in probability is at least 10%.

21. The use according to claim 16, wherein the rhSP-D is zelpultide alfa.

22. The use according to claim 16, wherein the rhSP-D is administered intratracheally, endotracheally, by inhalation, or by nebulization.

23. The use according to claim 16, wherein the premature infant at risk for developing BPD is an infant born at a gestational age of 29 weeks or less.

24. The use according to claim 16, wherein the premature infant at risk for developing BPD is an infant that requires respiratory support after birth.

25. The use according to claim 24, wherein the respiratory support is invasive respiratory support.

26. The use according to claim 25, wherein the invasive respiratory support comprises use of an endotracheal tube.

27. The use according to claim 16, wherein rhSP-D is administered at a dosage of from 1 milligram per kilogram (mg/kg) to 10 mg/kg per day for a time period of from 1 day to 10 days.
